(19) United States
(12) Patent Application Publication
(10) Pub. No.: US 2023/0263917 A1
(43) Pub. Date: Aug. 24, 2023

(54) STEAM BOOST SYSTEM FOR A STEAM STERILIZER

(71) Applicant: Indeeco, LLC, St. Louis, MO (US)

(72) Inventors: John G. Kozich, Cypress, TX (US); Scott Burkemper, O'fallon, MO (US); John D. Brune, Defiance, MO (US)

(21) Appl. No.: 18/085,221

(22) Filed: Dec. 20, 2022

Related U.S. Application Data
(60) Provisional application No. 63/312,577, filed on Feb. 22, 2022.

(51) Int. Cl. A61L 2/07; A61L 2/24; F22G 1/16

(52) U.S. Cl. CPC A61L 2/07; A61L 2/24; F22G 1/165; A61L 2202/14; H05B 3/40

(57) ABSTRACT

A steam boost system is communicated with a steam sterilizer and is operable to adjust a temperature of steam communicated from a source of steam to the steam sterilizer to a dry vapor superheated steam that prevents the occurrence of liquid moisture in the steam sterilizer.

### CROSS-REFERENCE TO RELATED APPLICATIONS

This patent application claims the benefit of the filing date of provisional patent application No. 63/312,577, which was filed on Feb. 22, 2022, the entire contents of which are incorporated herein by reference.

### STATEMENT REGARDING FEDERALLY SPONSORED RESEARCH OR DEVELOPMENT

Not Applicable.

### BACKGROUND

Medical devices such as surgical instruments and dental instruments are often stored in specialized packaging prior to the instruments being sterilized in an autoclave device. In the autoclave the instruments contained in the packaging go through the sterilization process of the autoclave by being subjected to saturated steam under pressure.

The autoclave steam sterilization process of the packaged instruments can at times result in moisture forming in the packaging, or a “wet pack”. A wet pack is a common industry problem in autoclave sterilization of medical and dental instruments and other sterile equipment. The occurrence of wet packs requires that the autoclave sterilization equipment be reprocessed, which is very costly. The occurrence of wet packs can be caused by the quality of steam supplied to the autoclave sterilization equipment, by equipment malfunction, and/or operator error.

In the food processing industry, food processing equipment and food packaging equipment are routinely sterilized in conjunction with the routine maintenance of the equipment to prevent and eliminate bacteria and other pathogens. In the steam sterilization of the food processing and food packaging equipment wet surfaces can occur where bacteria can form and propagate.

### BRIEF SUMMARY

What is needed to eliminate the problem of the occurrence of “wet packs” in autoclave sterilizers, the occurrence of wet surfaces when sterilizing food processing and packaging equipment, and in general the occurrence of “wet” surfaces or the presence of liquid moisture in steam sterilizers is a system of boosting the steam used in the sterilizers to dry vapor steam or superheated steam and thereby prevent the presence of liquid moisture in the sterilizers.

A steam boost system of this disclosure is operable to boost the temperature of steam used in a steam sterilizer to elevate the steam to dry vapor steam or superheated steam and prevent “wet” steam. The steam boost system is designed to be easily made a pat of the steam sterilizer or retrofit to an existing steam sterilizer. This is made possible by the steam boost system employing electric heating elements to heat and elevate the steam temperature. The steam boost system can be employed with a variety of different types of steam sterilizers. For example, the steam boost system can be used with an autoclave steam sterilizer to prevent the occurrence of “wet pack” in the autoclave steam sterilization process. The steam boost system can be used with a steam-cleaning apparatus for food processing equipment to prevent the occurrence of wet surfaces or liquid moisture on the food processing equipment. The steam boost system can be used with any other equivalent type of steam sterilizer where dry vapor steam or superheated steam is required.

The steam boost system is operatively communicated with a steam sterilizer. The steam boost system can be a component part of the steam sterilizer or can be retrofit to an existing steam sterilizer. In one example, the steam boost system is operatively communicated with a conventional autoclave. The autoclave is configured to contain equipment such as surgical instruments, dental instruments, etc. and more specifically the equipment contained in a sterilization packaging. In another example, the steam boost system is operatively communicated with a conventional steam sterilizer of food processing equipment. Again, the steam boost system can be a component part of the food processing equipment or can be retrofit to the equipment. Because the steam boost system of this disclosure employs electric heating elements to adjust a temperature of steam, the steam boost system can be a part of various different types of steam sterilization apparatus or can be retrofit to various different types of steam sterilization apparatus.

The steam boost system comprises a boost vessel or boost container. The container has a sealed container interior volume. The container interior volume has a length that extends through the boost container length. There is an inlet on the boost container. The inlet is positioned at one end or a first end of the boost container length. The inlet communicates with the interior volume of the boost container. There is an outlet on the boost container. The outlet is positioned on the boost container at the opposite end or a second end of the boost container length from the inlet. The outlet also communicates with the interior volume of the boost container.

An optional inlet temperature sensor is on the inlet of the boost container. The inlet temperature sensor is operable to sense a temperature of steam that is communicated from a supply of steam to the boost container as the steam passes through the inlet and into the boost container interior volume.

An outlet temperature sensor is on the outlet of the boost container. The outlet temperature sensor is operable to sense a temperature of steam in the interior volume of the boost container that is communicated from the interior volume of the boost container out through the outlet of the boost container as the steam passes through the outlet.

An optional pressure sensor is operatively connected to the boost container. The pressure sensor communicates with the interior volume of the boost container and is operable to sense a pressure of steam in the interior volume of the boost container.

The steam boost system is connected in the line of steam communication between a source of steam or a supply of steam and the steam sterilizer. The steam provided by the source of steam is typically saturated steam.

A supply conduit communicates the source of steam with the steam sterilizer. The supply conduit is a steam supply pipe or tube or other equivalent structure that functions as the line of steam communication between the source of steam and the steam sterilizer.

The supply conduit has a first, inlet end of the supply conduit. The first, inlet end of the supply conduit is operatively connected in fluid communication with the source of steam and with the inlet of the boost container.

The supply conduit also has a second, outlet end of the supply conduit. The second, outlet end of a supply conduit is operatively connected in fluid communication with the outlet of the boost container and with the steam sterilizer.

A heat conductor is contained in the interior volume of the boost container. The heat conductor has heating elements that extend through the length of the interior volume of the boost container. The heating elements are electrical resistance heating elements or heat transfer tubing. More specifically, the electric resistance heating elements are electric resistance tubular heating elements. The heating elements could be other equivalent types of electric heating elements.

A heat generator or a source of heat is operatively connected to the heat conductor, and more specifically to the electric heating elements of the heat conductor. The source of heat is operable to produce heat in the electric heating elements and to adjust the heat of the electric heating elements.

The steam boost system also includes a temperature control. The temperature control is operatively communicated with the inlet temperature sensor, the outlet temperature sensor, the pressure sensor, and the source of heat. The temperature control is operable to adjust or change electric current to the electric heating elements and thereby adjust a heat of the electric heating elements in the interior volume of the boost container in response to signals representing the temperature of steam passing through the outlet received from the outlet temperature sensor. The temperature control adjusts the heat of the electric resistance tubular heating elements to heat the steam passing through the boost container and maintain the steam at a desired temperature that ensures dry vapor steam or superheated steam is supplied to the steam sterilizer thereby preventing the occurrence of liquid moisture in the steam sterilizer.

### BRIEF DESCRIPTION OF THE DRAWINGS

FIG. 1 is a representation of a side, elevation view of the boost container, the inlet of the boost container, the outlet of the boost container and the source of heat.

FIG. 2 is a schematic representation of the boost container, the inlet of the boost container, the inlet temperature sensor on the inlet of the boost container, the outlet of the boost container, the outlet temperature sensor on the outlet of the boost container, the pressure sensor, the first inlet end of the supply conduit, the second outlet end of the supply conduit, the source of steam, the steam sterilizer and the temperature control.

Corresponding reference numerals will be used throughout the several figures of the drawings.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what is presently believed to be the best mode of carrying out the claimed invention. Additionally, it is to be understood that the claimed invention is not limited in its application to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

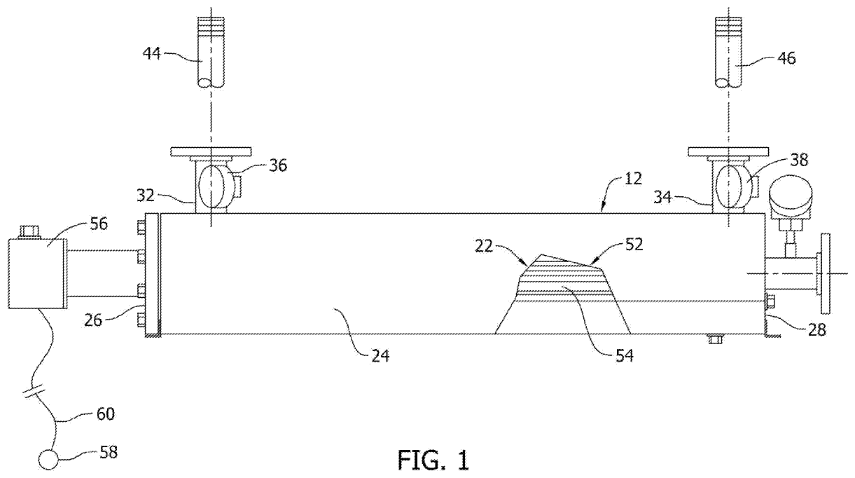

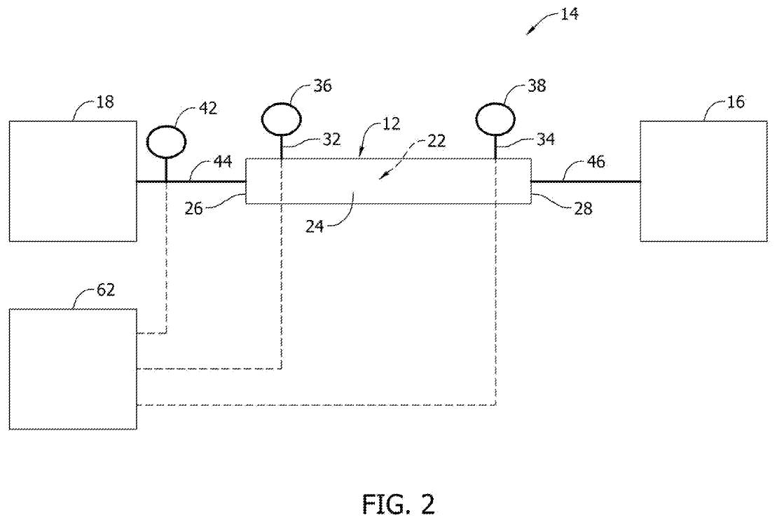

FIG. 1 is a representation of a side elevation view of the boost container 12 of the steam boost system 14 of this disclosure. FIG. 2 is a schematic representation of the steam boost system 14 used with a steam sterilizer 16, for example an autoclave steam sterilizer, a food processing equipment steam sterilizer or another equivalent type of steam sterilizer.

As represented in FIG. 2, the steam boost system 14 is comprised of a boost container 12, a steam sterilizer 16 and a source or supply of steam 18. The source or supply of steam 18 is operatively connected with the steam sterilizer 16 and operatively communicates steam with the steam sterilizer 16. The boost container 12 has an interior volume 22 that operatively communicates with the steam communicated from the source or supply of steam 18 to the steam sterilizer 16. The boost container 12, as well as the other component parts of the steam boost system 14 are constructed of corrosion resistant materials such as corrosion resistant metal alloys or other equivalent materials.

The source or supply of steam 18 could be any conventional source or supply of steam used with a steam sterilizer. The source or supply of steam 18 could be a separate component from the steam sterilizer 16, or a component part of the steam sterilizer 16. The steam provided by the source or supply of steam 18 is saturated steam.

The steam sterilizer 16 could be any conventional type of steam sterilizer. For example, the steam sterilizer 16 is a tank of an autoclave steam sterilizer. The steam sterilizer 16 can also be a steam-cleaning or sanitizing apparatus of food processing equipment or any other type of steam sterilizer.

As represented in FIG. 1, the boost container 12 is a circulation heater constructed as a cylindrical, sealed vessel comprising a cylindrical exterior housing 24. The cylindrical housing 24 has a length that extends between a first end or input end 26 of the housing to a second end or output end 28 of the housing. The sealed interior volume 22 of the boost container 12 also has a length that extends between the first end 26 of the housing to the second end 28 of the housing.

A first nozzle or inlet nozzle 32 is provided on the boost container housing 24. The first nozzle or inlet nozzle 32 functions as an inlet 32 on the boost container 12 adjacent the first end 26 of the boost container housing 24. The inlet 32 communicates with the interior volume 22 of the boost container housing 24.

A second nozzle or outlet nozzle 34 is provided on the boost container housing 24. The second nozzle or outlet nozzle 34 functions as an outlet 34 on the boost container 12 adjacent the second end 28 of the boost container housing 24. The outlet 34 communicates with the interior volume 22 of the boost container housing 24.

An inlet temperature sensor 36 is mounted on the first, inlet nozzle 32. The inlet temperature sensor 36 is operable to sense a temperature of steam passing through the first, inlet nozzle 32 and into the interior volume 22 of the boost container 12. The inlet temperature sensor 36 is preferably an electronic temperature sensor but could be any other equivalent type of temperature sensor.

An outlet temperature sensor 38 is mounted on the second, outlet nozzle 34. The outlet temperature sensor 38 is operable to sense a temperature of steam passing from the interior volume 22 of the boost container 12 out through the second, outlet nozzle 34. The outlet temperature sensor 38 is preferably an electronic temperature sensor. The outlet temperature sensor 38 could be any other equivalent type of temperature sensor.

A pressure sensor 42 is operatively communicated with the interior volume 22 of the boost container 12. As represented in FIG. 2, the pressure sensor 42 is physically separate from the boost container housing 24. However, the pressure sensor 42 could be operatively connected to the boost container housing 24 and could be directly mounted on the boost container housing 24. The pressure sensor 42 is operable to sense a pressure of steam communicating to and passing through the first, inlet nozzle 32 into the interior volume 22 of the boost container 12. The pressure sensor 42 is preferably an electronic sensor. The pressure sensor 42 could be any other equivalent type of pressure sensor.

A steam supply conduit communicates the source or supply of steam 18 with the steam sterilizer 16. The steam supply conduit could be constructed of steam supply pipes or tubing, or other equivalent structures. As represented in FIG. 2, the boost container 12 is connected in the line of the steam supply conduit between the source or supply of steam 18 and the steam sterilizer 16.

As represented in FIG. 2, the steam supply conduit is comprised of a first, inlet end of the supply conduit 44. The first, inlet end of supply conduit 44 is operatively connected in fluid communication with the source or supply of steam 18 and with the first, inlet nozzle 32 on the boost container 12. The first, inlet end of the supply conduit 44 is operable to directly communicate steam from the source or supply of steam 18 to the first, inlet nozzle 32.

The steam supply conduit is also comprised of a second, outlet end of the supply conduit 46. The second, outlet end of the supply conduit 46 is operatively connected in fluid communication with the second, outlet nozzle 34 on the boost container 12 and with the steam sterilizer 16. The second, outlet end of the supply conduit 46 is operable to directly communicate steam from the second, outlet nozzle 34 to the steam sterilizer 16.

The boost container 12 contains a heat conductor 52 in the interior volume 22 of the boost container 12. The heat conductor 52 comprises heating elements 54 that extend through the length of the interior volume 22 of the boost container 12. The heating elements 54 of the heat conductor 52 represented in FIG. 1 are electrical heating elements in the form of metal rods or metal tubes. More specifically, the electrical heating elements are electric resistance tubular heating elements 54 having elongate U-shaped or bent configurations that extend through the length of the interior volume 22 of the boost container 12. The configurations of the heating elements 54 extend from the input end 26 of the container 12, through the interior volume 22 of the container 12 to the bent ends of the heating elements positioned adjacent the output end 28 of the container 12.

An electric current control device 56 that functions as a source of electricity or electric power 56 is operatively connected to the heat conductor 52, and more specifically to the electric resistance tubular heating elements 54 of the heat conductor 52. The electric current control device 56 is operatively communicated with a source of electricity 58 by an electrical conductor 60. The source of electricity 58 and the electrical conductor 60 could be as simple as an electric wall outlet and an electrically conductive cord with an outlet plug, respectively. The electric current control device 56 is an electrical device that is operable supply an electric current to the electric resistance tubular heating elements to produce heat in the heating elements 54 and to adjust the heat of the heating elements 54 by resistance heating. By adjusting the heat of the electric resistance tubular heating elements 54, the electric current control device 56 is operable to adjust the temperature of steam in the interior volume 22 of the boost container 12 that comes into direct contact with the exterior surfaces of the heating elements 54. The resistance heating of the heating elements 54 enables the exterior surfaces of the heating elements 54 to transfer heat directly to the steam surrounding and contacting the exterior surfaces of the heating elements 54 in the interior volume 22 of the container 12 and heat the steam to produce dry vapor steam or superheated steam in the interior volume 22 of the container 12.

As represented in FIG. 2, the steam boost system 14 also comprises a temperature control 62. The temperature control 62 is operatively communicated with the inlet temperature sensor 36, the outlet temperature sensor 38 the pressure sensor 42 and the source of heat 56. The temperature control 62 is operable to receive electric signals from the inlet temperature sensor 36 and outlet temperature sensor 38 that are indicative or representative of the temperature of steam received by the boost container 12 from the source or supply of steam 18 and delivered from the boost container 12 to the steam sterilizer 16. The temperature control 62 also receives electric signals from the pressure sensor 42 that are indicative of the pressure of the steam passing through the boost container 12. In response to electric signals received from the inlet temperature sensor 36, the outlet temperature sensor 38 and the pressure sensor 42, the temperature control 62 is operable to control the electric current control device 56 and the heat conductor 52 to adjust the temperature of the electric resistance tubular heating elements 54 and thereby heat the steam and adjust the temperature of the steam passing through the boost container 12 to produce dry vapor steam or superheated steam. The temperature control 62 of the electric current control device 56 thereby maintains the steam passing through the boost container 12 and delivered to the steam sterilizer 16 at a desired temperature of dry vapor steam or superheated steam that prevents a “wet pack” occurrence or liquid moisture occurrence in the steam sterilizer 16.

Because the steam boost system is an electric steam boost system, the system can be easily and economically made a part of or retrofit to a steam sterilizer and a source of steam. The only requirement for the system to be added to a steam sterilizer is a source of electric power which could be as simple as a conventional electric outlet, making the steam boost system very versatile.

In view of the above, it will be seen that the several objects and advantages of the present invention have been achieved and other advantageous results have been obtained.

As various changes could be made in the above constructions of the steam boost system for a steam sterilizer without departing from the scope of the invention, it is intended that all matter contained in the above description or shown in the accompanying drawings shall be interpreted as illustrative and not in a limiting sense.

1. A steam boost system for a steam sterilizer comprising:
a steam sterilizer;
a source of steam operatively communicating steam from the source of steam to the steam sterilizer; and
a boost container operatively communicating with steam communicating from the source of steam to the steam sterilizer, the boost container being operable to sense a temperature of the steam communicating from the source of steam to the steam sterilizer and adjust a temperature of the steam by electric heating the steam to a dry vapor superheated steam and communicating the dry vapor superheated steam to the steam sterilizer.

2. The steam boost system of claim 1, further comprising:
the steam sterilizer is an autoclave steam sterilizer.

3. The steam boost system of claim 1, further comprising:
the steam sterilizer is a sterilizing apparatus of food processing equipment.

4. The steam boost system of claim 1, further comprising:
the boost container being a separate component from the steam sterilizer and is retrofit to the steam communicating from the source of steam to the steam sterilizer.

5. The steam boost system of claim 1, further comprising:
the boost container having an interior volume communicating with the steam communicating from the source of steam to the steam sterilizer, the boost container being operable to adjust a temperature of steam in the interior volume of the boost container to a dry vapor superheated steam.

6. The steam boost system of claim 5, further comprising:
electric heating elements in the boost container, the heating elements being operable to come into direct contact with steam communicating from the source of steam to the steam sterilizer and adjust a temperature of the steam to a dry vapor superheated steam.

7. The steam boost system of claim 6, further comprising:
the electric heating elements are electric resistance tubular heating elements that come into direct contact with the steam communicating from the source of steam to the steam sterilizer and transfer heat to the steam producing dry vapor superheated steam.

8. The steam boost system of claim 7, further comprising:
the electric resistance tubular heating elements have exterior surfaces inside the boost container that come into direct contact with steam inside the boost container and produce dry vapor superheated steam from the steam inside the boost container that contacts the exterior surfaces.

9. The steam boost system of claim 8, further comprising:
the boost container directly communicating with the steam sterilizer, the boost container directly communicating dry vapor superheated steam from inside the boost container to the steam sterilizer.

10. A steam boost system on a steam sanitizer comprising:
a boost container having an interior volume;
an inlet nozzle on the boost container, the inlet nozzle communicating with a supply of steam and communicating the supply of steam to the interior volume of the boost container;
electric heating elements in the interior volume of the boost container, the electric heating elements being operable to heat the supply of steam in the interior volume of the boost container and produce dry vapor superheated steam in the interior volume; and
an outlet nozzle on the boost container, the outlet nozzle communicating with a steam sterilizer and communicating the dry vapor superheated steam in the interior volume of the boost container to the steam sterilizer.

11. The steam boost system of claim 10, further comprising:
the steam sterilizer is an autoclave steam sterilizer.

12. The steam boost system of claim 10, further comprising:
the steam sterilizer is a sterilizing apparatus of food processing equipment.

13. The steam boost system of claim 10, further comprising:
the electric heating elements being operable to come into direct contact with the supply of steam in the interior volume of the boost container and produce dry vapor superheated steam in the interior volume.

14. The steam boost system of claim 13, further comprising:
the electric heating elements are electric resistance tubular heating elements that come into direct contact with the supply of steam in the interior volume of the boost container and transfer heat to the supply of steam In the interior volume of the boost container producing dry vapor superheated steam.

15. The steam boost system of claim 14, further comprising:
the electric resistance tubular heating elements have exterior surfaces inside the boost container that come into direct contact with the supply of steam inside the boost container and produce dry vapor superheated steam from the supply of steam inside the boost container that contacts the exterior surfaces of the electric resistance tubular heating elements.

16. The steam boost system of the claim 15, further comprising:
the boost container directly communicating with the steam sterilizer, the boost container directly communicating dry vapor superheated steam from inside the boost container to the steam sterilizer.

17. A method of preventing liquid moisture in a steam sterilizer comprising:
communicating a boost container containing electric heating elements with steam communicating from a source of steam to a steam sterilizer;
adjusting a temperature of steam communicating with the boost container to a dry vapor superheated steam; and
communicating the dry vapor superheated steam from the boost container to the steam sterilizer thereby preventing liquid moisture in the steam sterilizer.

18. The method of claim 17, further comprising:
heating steam communicating with the boost container by controlling electric current to the electric heating elements inside the boost container directly contacting the steam communicating with the boost container and transferring heat to the steam producing dry vapor superheated steam.

19. The method of claim 17, further comprising:
communicating the boost container with a steam sterilizer of an autoclave.

20. The method of claim 17, further comprising:
communicating the boost container with sterilizing apparatus of food processing equipment.
